(19) United States
(12) Patent Application Publication
(10) Pub. No.: US 2022/0359089 A1
(43) Pub. Date: Nov. 10, 2022

(54) Pandemic Community Tokenzation Tracing

(71) Applicant: International Business Machines Corporation, Armonk, NY (US)

(72) Inventors: Usman Basha Shaik, Bangalore (IN); Sriharidatta Sriharidatta, Mysore (IN); Anil Kumar Rajendran, Kollegal (IN)

(21) Appl. No.: 17/307,225

(22) Filed: May 4, 2021

(51) Int. Cl. G16H 50/80; G16H 70/60

(52) U.S. Cl. CPC G16H 50/80; G16H 70/60

(57) ABSTRACT

A mechanism is provided in a computing device for pandemic community tokenization tracing. The mechanism determines whether a user of the computing device has symptoms of an infectious disease based on readings from one or more sensors. The mechanism generates a symptomatic pandemic token in response to determining the user has symptoms of the infectious disease. The mechanism transmits the symptomatic pandemic token from the computing device to a central data processing system for analysis and broadcasts the symptomatic pandemic token via a short distance communication component of the computing device.

### BACKGROUND

The present application relates generally to an improved data processing apparatus and method and more specifically to mechanisms for pandemic community tokenization tracing.

Contact tracing is the process of identifying, notifying, and monitoring anyone who came in close contact with an individual who tested positive for an infectious disease while they were infectious. Contact tracing is a key strategy for preventing the further spread of infectious diseases, such as COVID-19. Close contacts of a case are considered to have been exposed to the infectious disease, and those close contacts may go on to develop the disease. Identifying and quarantining close contacts limits their ability to spread disease should they become infectious and helps to limit community spread.

### SUMMARY

This Summary is provided to introduce a selection of concepts in a simplified form that are further described herein in the Detailed Description. This Summary is not intended to identify key factors or essential features of the claimed subject matter, nor is it intended to be used to limit the scope of the claimed subject matter.

In one illustrative embodiment, a method is provided in a computing device for pandemic community tokenization tracing. The method comprises determining whether a user of the computing device has symptoms of an infectious disease based on readings from one or more sensors. The method further comprises generating a symptomatic pandemic token in response to determining the user has symptoms of the infectious disease. The method further comprises transmitting the symptomatic pandemic token from the computing device to a central data processing system for analysis. The method further comprises broadcasting the symptomatic pandemic token via a short distance communication component of the computing device.

In other illustrative embodiments, a computer program product comprising a computer useable or readable medium having a computer readable program is provided. The computer readable program, when executed on a computing device, causes the computing device to perform various ones of, and combinations of, the operations outlined above with regard to the method illustrative embodiment.

In yet another illustrative embodiment, a system/apparatus is provided. The system/apparatus may comprise one or more processors and a memory coupled to the one or more processors. The memory may comprise instructions which, when executed by the one or more processors, cause the one or more processors to perform various ones of, and combinations of, the operations outlined above with regard to the method illustrative embodiment.

These and other features and advantages of the present invention will be described in, or will become apparent to those of ordinary skill in the art in view of, the following detailed description of the example embodiments of the present invention.

### BRIEF DESCRIPTION OF THE DRAWINGS

The invention, as well as a preferred mode of use and further objectives and advantages thereof, will best be understood by reference to the following detailed description of illustrative embodiments when read in conjunction with the accompanying drawings, wherein:

FIG. 1 is an example diagram of a distributed data processing system in which aspects of the illustrative embodiments may be implemented;

FIG. 2 is an example block diagram of a computing device in which aspects of the illustrative embodiments may be implemented;

FIG. 3 is a block diagram of one example of a user device in which aspects of the illustrative embodiments may be implemented;

FIG. 4 is a state flow diagram of a user of the pandemic tokenization tracing system in accordance with an illustrative embodiment;

FIG. 5 illustrates pandemic token generation, transmission, and tracing in accordance with an illustrative embodiment;

FIG. 6 illustrates an example pandemic token in accordance with an illustrative embodiment;

FIG. 7 illustrates token tree traversal in accordance with an illustrative embodiment;

FIGS. 8A-8C are flowcharts illustrating operation of a user device for pandemic community tokenization tracing in accordance with an illustrative embodiment; and

FIG. 9 is a flowchart illustrating operation of a central data processing system for pandemic community tokenization tracing in accordance with an illustrative embodiment.

### DETAILED DESCRIPTION

A major challenge in pandemic community spread is identifying asymptomatic carriers. These carriers may not be aware that they are spreading an infectious disease. When an infected person is identified, the tracing and stopping of the spread is a major challenge. Tracing with travel history is one common approach but depends on the memories of the carrier. The data gathered using these traditional approaches requires detailed analysis to identify the correct persons who are now carriers.

Using current approaches, manual updating of personal health is left to the individuals. Many do not update their health records due to fear and social stigma. This leads to inaccurate or ineffective analysis, resulting in a failure to identify nodes representing asymptomatic infectious disease carriers. Inaccurate or incomplete updating of health and travel records also leads to an inability to accurately identify zones with more infected individuals and create alerts to avoid contamination zones to avoid spreading.

The illustrative embodiments overcome the challenges of traditional approaches by providing mechanisms for pandemic token creation and exchange. The mechanisms of the illustrative embodiment provide three types of pandemic tokens: symptomatic, social distance, and geographical code (geo code).

In accordance with one illustrative embodiment, user devices include sensors for detecting symptoms on a user's personal device. In response to the sensors indicating the user is symptomatic of an infectious disease, the personal device creates a “symptomatic” pandemic token. In one example embodiment, the devices have sensors to capture thermal readings and a peripheral capillary oxygen saturation (also referred to as SpO2 or pulse oximetry) sensor to detect symptoms of COVID-19 infection. The thermal sensor is used to detect when the user has a fever (internal temperature over a predetermined threshold) consistently for a predetermined period of time. The SpO2 sensor provides an estimate of the amount of oxygen in the blood. The SpO2 sensor is used to detect when the amount of oxygen in the blood falls below a predetermined threshold.

In accordance with an illustrative embodiment, user devices include a short distance communication component (transmitter/receiver) for communicating pandemic tokens. The short distance communication components are tuned to social distance restrictions during a pandemic. For instance, for COVID-19, social distance measures may recommend a six-foot, ten-foot, one-meter, or two-meter distance between people, depending on the state, country, or agency. In one example embodiment, the short distance communication component uses the Industrial, Scientific, and Medical (ISM) band, which are portions of the radio spectrum reserved internationally for industrial, scientific, and medical (ISM) purposes other than telecommunications. ISM band is only a medium of detection for the proximity of people. This can be any frequency mandated by authorized agencies during a pandemic. In the United States, uses of the ISM bands are governed by Part 18 of the Federal Communications Commission (FCC) rules, while Part 15 contains the rules for unlicensed communication devices, even those that share ISM frequencies. In Europe, the ETSI is responsible for regulating the use of short-range devices, some of which operation in ISM bands. A user's device creates a “social distance” pandemic token in response to receiving a symptomatic token from another device via the short distance communication component. In other words, when two user devices are near enough to communicate via the short distance communication components, then it is assumed that social distancing has been violated, and the devices exchange pandemic tokens.

In accordance with one embodiment, pandemic token communication access points are placed at various locations throughout the community. These pandemic token communication access points receive pandemic tokens from user devices and broadcast pandemic tokens to user devices that are within communication range via the short distance communication component. This embodiment assumes that visiting a location where symptomatic or asymptomatic carriers previously visited exposes one to the infectious disease. The user's device creates a “geo code” pandemic token in response to receiving a pandemic token (symptomatic and/or social distance) from the pandemic token communication access point.

The illustrative embodiments described herein may implement, and make use of, artificial intelligence (AI) and/or cognitive systems. The purpose of these AI and/or cognitive systems is to augment, not replace, human intelligence. These AI and/or cognitive systems are designed to enhance and extend human capabilities and potential through specific improved computer tools and improved computer tool operations. These improved computer tools perform operations at a speed, complexity, and volume that is not practically able to be performed by human intelligence. While such AI and/or cognitive systems may emulate achieving similar results to that of human intelligence, they do so using different methodologies and mechanisms specific to computer tools that are not the same as any mental processes or manual efforts of human beings due, at least in part, to the inherent differences in the way that computing devices operate from the way that human minds operate.

The systems implemented by the illustrative embodiments may operate on various types of data, which may include personal or private information of individuals. While the systems may operate on such personal or private information, the systems implement various mechanisms (not specifically shown in the figures) for maintaining the privacy and security of individual's personal or private information and implement a principle of trust and transparency regarding the security of such personal or private information. This principle of trust and transparency recognizes that any person whose data is tracked and shared should always be given the option to opt-in or opt-out of such tracking and sharing of their personal or private data. This principle of trust and transparency recognizes that a person whose data is tracked and shared should always have control over the use of the data, what entities have access to that data, and the ability to have that data deleted. Moreover, this principle of trust and transparency recognizes that a person's personal or private data should be kept secure from cyber threats and that such data should not be used for purposes, such as government tracking and surveillance, which are not specifically approved by the individual who, again, is the ultimate owner of this personal and/or private data.

Thus, where the systems operate on any such personal or private information, these mechanisms implement functionality for individuals to opt-in or opt-out of usage of their personal/private data, authorize entities to access their personal/private data, and provide security mechanisms to ensure that the individual's personal/private data is secure from cyber threats. These mechanisms do not require individuals to relinquish ownership rights in their personal/private data or insights derived from the personal/private data to have benefit of the illustrative embodiments. While the illustrative embodiments may promote and utilize free movement of data across one or more data networks which may span organizational and geopolitical borders, such free movement of data is done so using mechanisms that promote security of the personal/private data flows.

The illustrative embodiments provide a data processing system that collects tokens from the personal devices and creates a trace tree data structure to identify and navigate the carriers from any nodes in the tree data structure. The data processing system may include a medical application and/or government agency application to analyze the pandemic token data. The data processing system may identify clusters of nodes in the trace tree data structure, identify and declare contamination zones, and notify nodes to recommend quarantine. The data processing system generates and outputs a graphical user interface (GUI) with analysis output. In the illustrative embodiments, the data processing system implements mechanisms that strip out or obfuscate personal or private information (e.g., International Mobile Equipment Identity (IMEI) number of the user's device) from token data provided to a government agency. This allows a government agency to analyze pandemic token data to identify contamination zones or establish trends without tying the tokens to any individuals.

Before beginning the discussion of the various aspects of the illustrative embodiments and the improved computer operations performed by the illustrative embodiments, it should first be appreciated that throughout this description the term “mechanism” will be used to refer to elements of the present invention that perform various operations, functions, and the like. A “mechanism,” as the term is used herein, may be an implementation of the functions or aspects of the illustrative embodiments in the form of an apparatus, a procedure, or a computer program product. In the case of a procedure, the procedure is implemented by one or more devices, apparatus, computers, data processing systems, or the like. In the case of a computer program product, the logic represented by computer code or instructions embodied in or on the computer program product is executed by one or more hardware devices to implement the functionality or perform the operations associated with the specific “mechanism.” Thus, the mechanisms described herein may be implemented as specialized hardware, software executing on hardware to thereby configure the hardware to implement the specialized functionality of the present invention which the hardware would not otherwise be able to perform, software instructions stored on a medium such that the instructions are readily executable by hardware to thereby specifically configure the hardware to perform the recited functionality and specific computer operations described herein, a procedure or method for executing the functions, or a combination of any of the above.

The present description and claims may make use of the terms “a”, “at least one of”, and “one or more of” regarding particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

Moreover, it should be appreciated that the use of the term “engine,” if used herein regarding describing embodiments and features of the invention, is not intended to be limiting of any particular implementation for accomplishing and/or performing the actions, steps, processes, etc., attributable to and/or performed by the engine. An engine may be, but is not limited to, software, hardware and/or firmware or any combination thereof that performs the specified functions including, but not limited to, any use of a general and/or specialized processor in combination with appropriate software loaded or stored in a machine-readable memory and executed by the processor. Further, any name associated with a particular engine is, unless otherwise specified, for purposes of convenience of reference and not intended to be limiting to a specific implementation. Additionally, any functionality attributed to an engine may be equally performed by multiple engines, incorporated into and/or combined with the functionality of another engine of the same or different type, or distributed across one or more engines of various configurations.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the examples provided herein without departing from the spirit and scope of the present invention.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

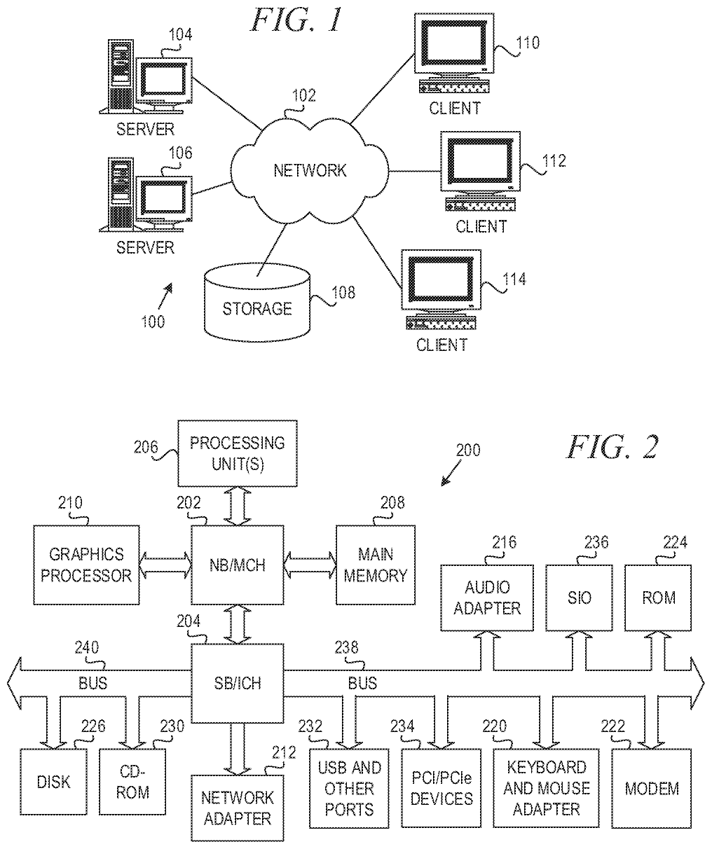

The illustrative embodiments may be utilized in many different types of data processing environments. In order to provide a context for the description of the specific elements and functionality of the illustrative embodiments, FIGS. 1 and 2 are provided hereafter as example environments in which aspects of the illustrative embodiments may be implemented. It should be appreciated that FIGS. 1 and 2 are only examples and are not intended to assert or imply any limitation regarding the environments in which aspects or embodiments of the present invention may be implemented. Many modifications to the depicted environments may be made without departing from the spirit and scope of the present invention.

FIG. 1 depicts a pictorial representation of an example distributed data processing system in which aspects of the illustrative embodiments may be implemented. Distributed data processing system 100 may include a network of computers in which aspects of the illustrative embodiments may be implemented. The distributed data processing system 100 contains at least one network 102, which is the medium used to provide communication links between various devices and computers connected within distributed data processing system 100. The network 102 may include connections, such as wire, wireless communication links, or fiber optic cables.

In the depicted example, server 104 and server 106 are connected to network 102 along with storage unit 108. In addition, clients 110, 112, and 114 are also connected to network 102. These clients 110, 112, and 114 may be, for example, personal computers, network computers, or the like. In the depicted example, server 104 provides data, such as boot files, operating system images, and applications to the clients 110, 112, and 114. Clients 110, 112, and 114 are clients to server 104 in the depicted example. Distributed data processing system 100 may include additional servers, clients, and other devices not shown.

In the depicted example, distributed data processing system 100 is the Internet with network 102 representing a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational, and other computer systems that route data and messages. Of course, the distributed data processing system 100 may also be implemented to include a number of different types of networks, such as for example, an intranet, a local area network (LAN), a wide area network (WAN), or the like. As stated above, FIG. 1 is intended as an example, not as an architectural limitation for different embodiments of the present invention, and therefore, the particular elements shown in FIG. 1 should not be considered limiting with regard to the environments in which the illustrative embodiments of the present invention may be implemented.

As shown in FIG. 1, one or more of the computing devices, e.g., server 104, may be specifically configured to implement a data processing system and user devices for pandemic community tokenization tracing. The configuring of the computing device may comprise the providing of application specific hardware, firmware, or the like to facilitate the performance of the operations and generation of the outputs described herein regarding the illustrative embodiments. The configuring of the computing device may also, or alternatively, comprise the providing of software applications stored in one or more storage devices and loaded into memory of a computing device, such as server 104, for causing one or more hardware processors of the computing device to execute the software applications that configure the processors to perform the operations and generate the outputs described herein regarding the illustrative embodiments. Moreover, any combination of application specific hardware, firmware, software applications executed on hardware, or the like, may be used without departing from the spirit and scope of the illustrative embodiments.

It should be appreciated that once the computing device is configured in one of these ways, the computing device becomes a specialized computing device specifically configured to implement the mechanisms of the illustrative embodiments and is not a general-purpose computing device. Moreover, as described hereafter, the implementation of the mechanisms of the illustrative embodiments improves the functionality of the computing device and provides a useful and concrete result that facilitates pandemic community token tracing.

These computing devices, or data processing systems, may comprise various hardware elements which are specifically configured, either through hardware configuration, software configuration, or a combination of hardware and software configuration, to implement one or more of the systems/subsystems described herein. FIG. 2 is a block diagram of just one example data processing system in which aspects of the illustrative embodiments may be implemented. Data processing system 200 is an example of a computer, such as server 104 in FIG. 1, in which computer usable code or instructions implementing the processes and aspects of the illustrative embodiments of the present invention may be located and/or executed to achieve the operation, output, and external effects of the illustrative embodiments as described herein.

In the depicted example, data processing system 200 employs a hub architecture including north bridge and memory controller hub (NB/MCH) 202 and south bridge and input/output (I/O) controller hub (SB/ICH) 204. Processing unit 206, main memory 208, and graphics processor 210 are connected to NB/MCH 202. Graphics processor 210 may be connected to NB/MCH 202 through an accelerated graphics port (AGP).

In the depicted example, local area network (LAN) adapter 212 connects to SB/ICH 204. Audio adapter 216, keyboard and mouse adapter 220, modem 222, read only memory (ROM) 224, hard disk drive (HDD) 226, CD-ROM drive 230, universal serial bus (USB) ports and other communication ports 232, and PCI/PCIe devices 234 connect to SB/ICH 204 through bus 238 and bus 240. PCI/PCIe devices may include, for example, Ethernet adapters, add-in cards, and PC cards for notebook computers. PCI uses a card bus controller, while PCIe does not. ROM 224 may be, for example, a flash basic input/output system (BIOS).

HDD 226 and CD-ROM drive 230 connect to SB/ICH 204 through bus 240. HDD 226 and CD-ROM drive 230 may use, for example, an integrated drive electronics (IDE) or serial advanced technology attachment (SATA) interface. Super I/O (SIO) device 236 may be connected to SB/ICH 204.

An operating system runs on processing unit 206. The operating system coordinates and provides control of various components within the data processing system 200 in FIG. 2. As a client device, the operating system may be a commercially available operating system such as Microsoft® Windows 10®. An object-oriented programming system, such as the Java™ programming system, may run in conjunction with the operating system and provides calls to the operating system from Java™ programs or applications executing on data processing system 200.

As a server, data processing system 200 may be, for example, an IBM eServer™ System p® computer system, Power™ processor-based computer system, or the like, running the Advanced Interactive Executive (AIX®) operating system or the LINUX® operating system. Data processing system 200 may be a symmetric multiprocessor (SMP) system including a plurality of processors in processing unit 206. Alternatively, a single processor system may be employed.

Instructions for the operating system, the object-oriented programming system, and applications or programs are located on storage devices, such as HDD 226, and may be loaded into main memory 208 for execution by processing unit 206. The processes for illustrative embodiments of the present invention may be performed by processing unit 206 using computer usable program code, which may be located in a memory such as, for example, main memory 208, ROM 224, or in one or more peripheral devices 226 and 230, for example.

A bus system, such as bus 238 or bus 240 as shown in FIG. 2, may be comprised of one or more buses. Of course, the bus system may be implemented using any type of communication fabric or architecture that provides for a transfer of data between different components or devices attached to the fabric or architecture. A communication unit, such as modem 222 or network adapter 212 of FIG. 2, may include one or more devices used to transmit and receive data. A memory may be, for example, main memory 208, ROM 224, or a cache such as found in NB/MCH 202 in FIG. 2.

As mentioned above, in some illustrative embodiments the mechanisms of the illustrative embodiments may be implemented as application specific hardware, firmware, or the like, application software stored in a storage device, such as HDD 226 and loaded into memory, such as main memory 208, for executed by one or more hardware processors, such as processing unit 206, or the like. As such, the computing device shown in FIG. 2 becomes specifically configured to implement the mechanisms of the illustrative embodiments and specifically configured to perform the operations and generate the outputs described hereafter with regard to pandemic community tokenization tracing.

Those of ordinary skill in the art will appreciate that the hardware in FIGS. 1 and 2 may vary depending on the implementation. Other internal hardware or peripheral devices, such as flash memory, equivalent non-volatile memory, or optical disk drives and the like, may be used in addition to or in place of the hardware depicted in FIGS. 1 and 2. Also, the processes of the illustrative embodiments may be applied to a multiprocessor data processing system, other than the SMP system mentioned previously, without departing from the spirit and scope of the present invention.

Moreover, the data processing system 200 may take the form of any of a number of different data processing systems including client computing devices, server computing devices, a tablet computer, laptop computer, telephone or other communication device, a personal digital assistant (PDA), or the like. In some illustrative examples, data processing system 200 may be a portable computing device that is configured with flash memory to provide non-volatile memory for storing operating system files and/or user-generated data, for example. Essentially, data processing system 200 may be any known or later developed data processing system without architectural limitation.

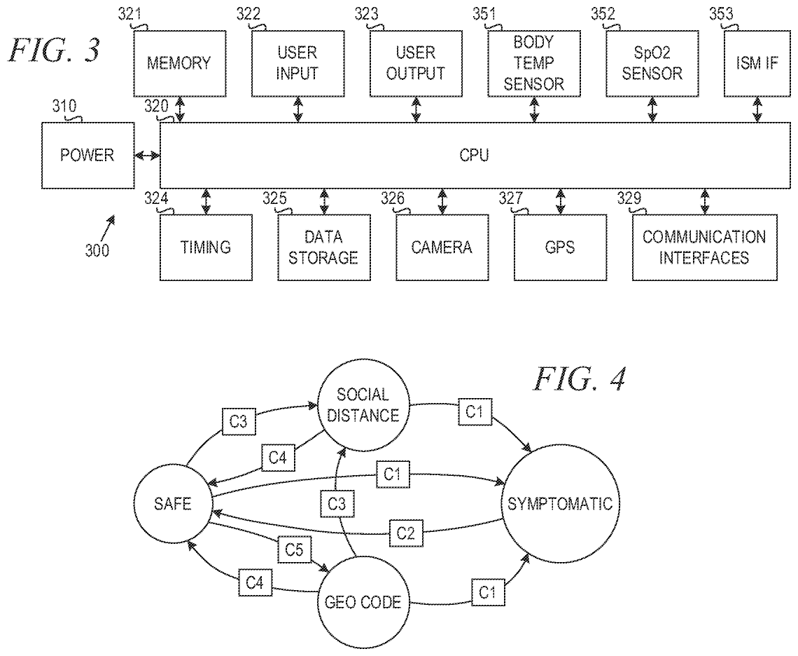

FIG. 3 is a block diagram of one example of a user device in which aspects of the illustrative embodiments may be implemented. Device 300 may be a smartphone, smartwatch, tablet, or similar portable device carried by the user and capable of receiving and installing software for carrying out the pandemic community tokenization tracing functions of the illustrative embodiments. Such software is known as a mobile app, although it is not limited to any particular programming language, operating system, or platform. FIG. 3 shows required components of the user device 300, including a hardware central processing unit (CPU) 320, memory 321, and data storage 325, and various components that may include or be implemented as hardware, programmed firmware, or software instructions for execution by the CPU 320. The components of user device 300 are powered by power supply 310, which may be a battery in one example embodiment.

The components include a user input interface 322, which may include a microphone, a touchscreen, a keypad, an orientation or motion sensor, or any other input device or combination of input devices, and a user output interface 323, which may include a display screen (such as the touchscreen), a speaker, and/or a vibration generating device. The user device also includes camera 326 and communication interfaces 329, including a transceiver, SIM card, and/or any other device or circuit that allows wireless communications, a timing system or clock 324, which may be included in the CPU, a global positioning system (GPS) interface 327, which may include circuitry for processing location signals from the cellular network or received by a global positioning system (GPS), global navigating satellite system (GNSS or GLONASS) transceiver as well as additional sensors, such as motion, acceleration, and/or sensors, and magnetometer or other sensors or devices (not shown) for determining an orientation or direction of the user device 300.

In accordance with the illustrative embodiment, user device 300 includes body temperature sensor 351 and SpO2 sensor 352 for detecting symptoms of the user. While not shown in FIG. 3, additional sensors may be included to measure other vitals depending on the pandemic hit or the virus affected. In one example embodiment, body temperature sensor 351 is an infrared sensor, often referred to as a forehead or temporal thermometer, which measures the temperature of the superficial temporal artery, which is a branch of the carotid artery. Body temperature sensor 351 may be a non-contact thermometer that requires no physical contact, which has become popular for use in venues such as airports, stores, and stadiums. SpO2 sensor 352 is a medical sensor that indirectly measures the oxygen saturation of the user's blood and changes in blood volume in the skin. Other sensors may be used in addition to or in place of body temperature sensor 351 and SpO2 sensor 352 depending on the infectious disease being traced and the corresponding symptoms being monitored.

In one embodiment, sensors 351, 352 are mounted on the user device, which is wearable by the user. Monitoring of readings from sensors 351, 352 may be mandated and enforced during a pandemic. Alternatively, participation may be voluntary and incentivized in some way. Software in memory 321 of user device 300 operates to take readings from sensors 351, 352. This software may be executed in a reserved memory space in memory 321 and utilize priority CPU cycles. This is to ensure that readings are captured at regular intervals throughout the day, week, etc. In the depicted example, the software monitors the body temperature and oxygen levels in the blood stream.

In accordance with the illustrative embodiment, user device 300 monitors the sensor data and compares this data to predetermined threshold. For example, user device 300 may compare body temperature from sensor 351 to a temperature threshold to detect when the temperature is greater than the threshold. As another example, user device 300 may compare the SpO2 reading from sensor 352 to a blood oxygen threshold to detect when the blood oxygen level is less than the threshold. User device 300 then determines when the user has symptoms of an infectious disease based on these comparisons. For instance, user device 300 may determine the user is potentially symptomatic and a carrier of infectious disease in response to the body temperature exceeding the temperature threshold and blood oxygen falling below the blood oxygen threshold consistently for a period of time (e.g., one hour).

In response to the user device 300 determining the user is potentially symptomatic, the user device 300 generates a “symptomatic” pandemic token. The symptomatic pandemic token may include a device identifier or user identifier. In one example embodiment, this identifier may be the International Mobile Equipment Identity (IMEI) number of the user device 300. Alternatively, this identifier may be a media access control (MAC) address or another device identifier. In another example embodiment, this identifier may be a personal identifier of the user, such as a social security number. In yet another embodiment, this identifier may be assigned to the user for the purpose of token tracing only such that the identifier cannot be traced back to an individual. The symptomatic pandemic token also includes the sensor readings, a location of the device, and a time stamp. In one embodiment, the identifier is encrypted or converted using a hash function to obfuscate personal or private information.

In accordance with the illustrative embodiment, user device 300 includes Industrial, Scientific, and Medical (ISM) communication interface 353. In the depicted example, ISM interface 353 has a short range, which can be tuned to social distancing measures of the infectious disease (e.g., six feet, ten feet, one meter, two meters, etc.). The user device 300 uses ISM interface 353 to send and receive pandemic tokens. In the case of a potentially symptomatic user, user device 300 broadcasts the symptomatic pandemic token to other user devices via ISM interface 353. This communication happens when the two user devices are within communication range of the ISM communication interface 353, i.e., when social distancing recommendations are violated. For instance, if the user of device 300 is asymptomatic and a symptomatic pandemic token is received via ISM interface 353, then device 300 generates a “social distance” pandemic token to represent that the user may be an asymptomatic carrier. In one example embodiment, a social distance pandemic token may include a root token identifier (identifier of symptomatic user's device), leaf token (identifier of user's device), sensor data placeholders in case user becomes symptomatic, a location of the user device, and a time stamp.

In one embodiment, device 300 may be a pandemic token communication access point for tracing locations that have been visited by symptomatic or asymptomatic carriers. In one example embodiment, device 300 may be embodied within a wireless fidelity (Wi-Fi) access point or other stationary communication device. In another example embodiment, device 300 may be one of many devices distributed throughout a community for monitoring and detecting hotspots of infectious disease transmission, such as businesses, community facilities, post offices, places of worship, stadiums, parks, and the like.

Many infectious diseases, such as COVID-19, are transmissible via droplets and/or aerosols. If a location has been visited by a symptomatic or asymptomatic carrier, then another person may be exposed via contact with surfaces or by breathing the air. When one or more symptomatic or asymptomatic carriers are within the communication range of ISM interface 353 of device 300, then the device 300 receives the pandemic token and assumes the infectious disease may be transmitted to other people who visit the location for a period of time. The device 300 may keep these pandemic tokens, or at least the most recent pandemic token, for a predetermined period of time and broadcast them to other user devices.

In response to a user device 300 receiving a pandemic token from a pandemic token communication access point via ISM interface 353, the user device 300 generates a geographic location or “geo code” pandemic token. In this embodiment, a geo code pandemic token is like a social distance pandemic token and represents a user who is potentially an asymptomatic carrier.

FIG. 4 is a state flow diagram of a user of the pandemic tokenization tracing system in accordance with an illustrative embodiment. Users start in a “safe” state representing a user who has no symptoms and is not a carrier of the infectious disease. In response to a first condition (C1) in which the user's device detects symptoms of the infectious disease, the user may transition from the “safe” state to the “symptomatic” state. In the “symptomatic” state, the user's device generates and transmits a symptomatic pandemic token. The user may return to the “safe” state from the “symptomatic” state in response to a second condition (C2) in which the user's device detects the user is not experiencing symptoms for a life span of the symptomatic token, meaning the user is no longer a symptomatic carrier. As an example, this symptomatic token life span may be set to two or three weeks from the time the user is not showing symptoms.

The user transitions from the “safe” state to a “social distance” state in response to a third condition (C3) in which the user's device detects that social distance recommendations have been violated. This may be determined based on receiving any pandemic token from another user's device. In the “social distance” state, the user's device generates and transmits a social distance pandemic token. The user may return to the “safe” state from the “social distance” state in response to a fourth condition (C4) in which the user remains asymptomatic and follows social distancing recommendations for a life span of the social distance pandemic token. The life span of the social distance pandemic token may be equal to the life span of the symptomatic token; however, the life span of a symptomatic token does not begin until the user is no longer showing symptoms. The user transitions from the “social distance” state to the “symptomatic state in response to the first condition (C1) in which the user's device detects symptoms of the infectious disease.

The user transitions from the “safe” state to a “geo code” state in response to a fifth condition (C5) in which the user's device detects that the user has visited a location previously visited by symptomatic or asymptomatic carriers. This may be determined based on receiving any pandemic token from pandemic token communication access point. In the “geo code” state, the user's device generates and transmits a geo code pandemic token. The user may return to the “safe” state from the “geo code” state in response to the fourth condition (C4) in which the user remains asymptomatic and follows social distancing recommendations for a life span of the social distance pandemic token. In the depicted example, the life span of the geo code pandemic token equal to the life span of the social distance token; however, the life spans may be different depending upon the implementation. The user transitions from the “geo code” state to the “symptomatic state in response to the first condition (C1) in which the user's device detects symptoms of the infectious disease. In the embodiment depicted in FIG. 4, the user may also transition from the “geo code” state to the “social distance” state in response to the third condition (C3) in which the user's device detects that social distance recommendations have been violated.

Although not shown, the user may also transition from the “social distance” state to the “geo code” state in response to the fifth condition (C5) in which the user's device detects that the user has visited a location previously visited by symptomatic or asymptomatic carriers. This may serve the purpose of resetting the life span of the pandemic token each time the user is exposed to the infectious disease. Similarly, the life span of the token may be reset while in the “social distance” or “geo code” state whenever the corresponding conditions are detected.

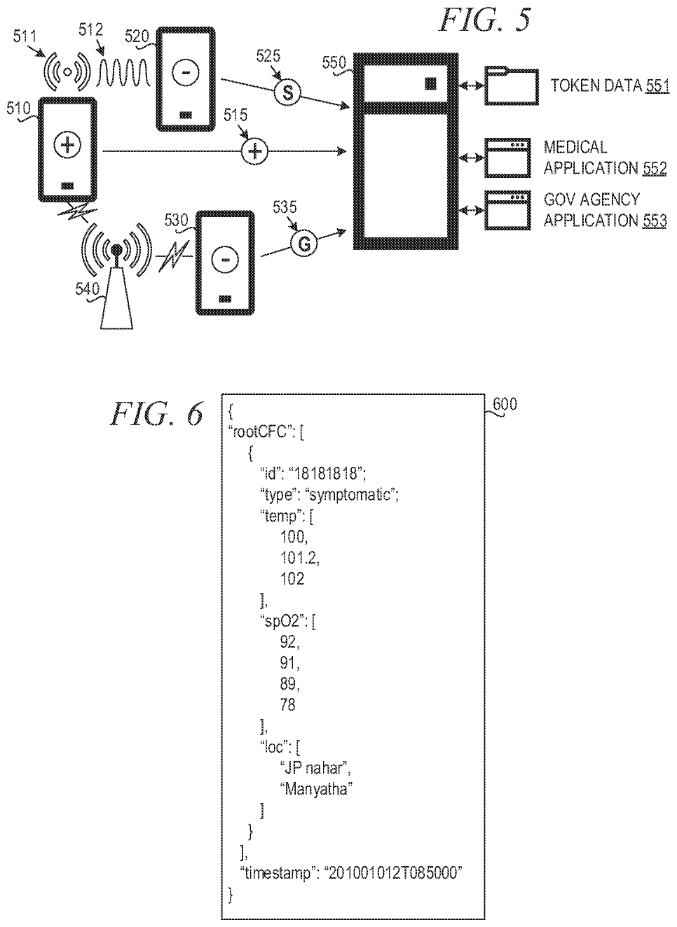

FIG. 5 illustrates pandemic token generation, transmission, and tracing in accordance with an illustrative embodiment. User device 510 detects that the user is symptomatic based on sensor readings. User device 510 generates a symptomatic pandemic token 515 and transmits the pandemic token 515 to central data processing system or server 550. User device 510 also broadcasts 511 the symptomatic token via ISM communication signal 512.

In the depicted example, user device 520 receives the symptomatic token from user device 510. In response, user device 520 generates a social distance token 525 and transmits the pandemic token 525 to central data processing system 550.

In accordance with one embodiment, user device 510 transmits the symptomatic pandemic token to pandemic token communication access point 540. When user device 530 is within communication range of pandemic token communication access point 540, user device 530 receives the symptomatic pandemic token or another token indicating the geographic location associated with the access point 540 is a potential location of infectious disease exposure. User device 530 then generates a geo code pandemic token 535 and transmits the pandemic token 535 to central data processing system 550.

The central data processing system 550 collects pandemic tokens and stores the token data 551. Central data processing system 550 strips the pandemic tokens and forms a tree data structure. Data processing system 550 extracts the root identifier and traverses the tree down to the left node. During traversal, each node is analyzed to determine whether the node matches the tree node. If any node is not matched until the last tree node is reached, the final position plus one is recorded. Data processing system 550 then traverses to the right nodes recursively to match nodes. If no match is found, then the node is inserted at the recorded position. If the node matches with the root node, then the leaf node of the token is extracted and attached to the resultant node.

Central data processing system 550 includes medical application 552 and/or government agency application 553. These applications 552, 553 identify clusters of nodes, identify high density locations, declare contamination zones, and identify nodes for quarantine. Applications 552, 553 may also generate a graphical user interface (GUI) for token analysis output.

FIG. 6 illustrates an example pandemic token in accordance with an illustrative embodiment. In the depicted example, pandemic token 600 is a symptomatic pandemic token. The token includes an IMEI number, pandemic token type, temperature and SpO2 sensor readings, location, and time stamp. In one example embodiment, the IMEI number or another identifier may be encrypted to obfuscate the user's identity for device-to-device transmission. In the case of a social distance token, the pandemic token may include a root or parent identifier.

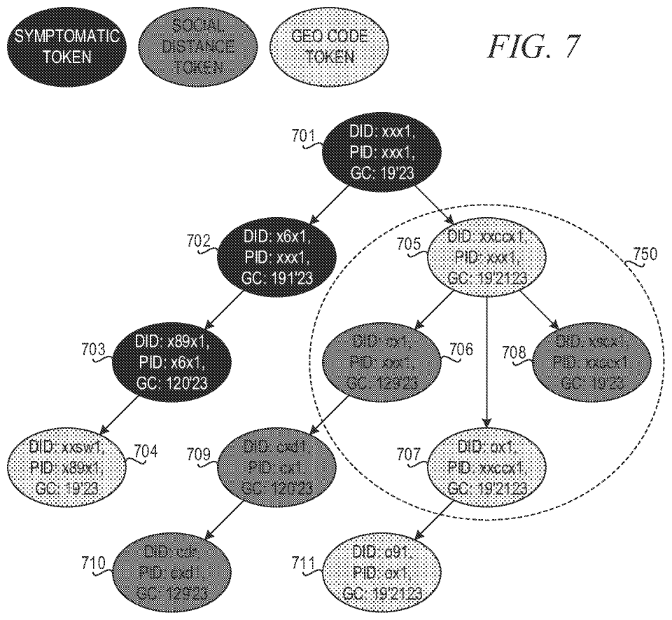

FIG. 7 illustrates token tree traversal in accordance with an illustrative embodiment. The token tree includes three types of pandemic tokens: symptomatic, social distance, and geo code. In this embodiment, each token has a device ID, parent ID, and encrypted geographic location attached.

The attach process navigates to the first node in the tree, node 701. The attach process then compares the token to the parent node, and if the token matches the parent node, the attach process attaches the token as a child of the parent node. If the token does not match, the attach process proceeds to the left node 702 and repeats this process. If there is no match for node 702, node 703, or node 704, then the attach process checks on the right, by back tracking as −1, to take one step above and navigate to the right.

Thus, in the example shown in FIG. 7, the attach process navigates up to node 701 and traverses to the right to node 704. The attach process then repeats the process by traversing to the left from node 704 to node 706, and so on. If there is no match for node 706, node 709, or node 710, then the attach process checks on the right to take one step above and navigate to the right. Thus, in the example shown in FIG. 7, the attach process navigates up to node 705 and traverses to the right to node 707. The attach process then repeats the process by traversing to the left from node 711. Then, the attach process navigates up to node 705 and traverses to the right to node 708.

As more nodes become attached to a parent node, such as node 705, a cluster 750 may be identified. Clusters are high-dense zones where the infection is too high, which would need special attention, probably redirection of medical care and other resources. Also, clusters will be marked so that anyone visiting such a location is restricted or directed to take precaution if the visit is unavoidable.

A symptomatic pandemic token has a life span that expires when the token is marked as fixed. For example, the symptomatic pandemic token may have a life span that expires a predetermined number of days after the user is no longer showing symptoms. The user device may send notification to the central data processing system indicating that a symptomatic pandemic token is fixed.

Social distance pandemic tokens and geo code pandemic tokens are prospect infectants. The life span of these tokens may have a fixed time value (e.g., two weeks), which is configurable based on the infectious disease and may be defined by a government agency, such as the World Health Organization (WHO). If the token is not changed to symptomatic before the life span expires, then the token may be deleted.

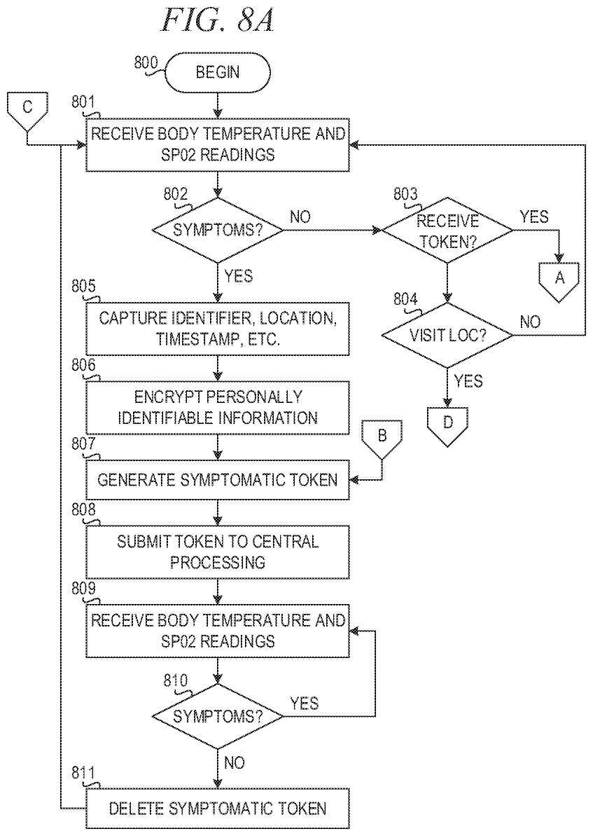

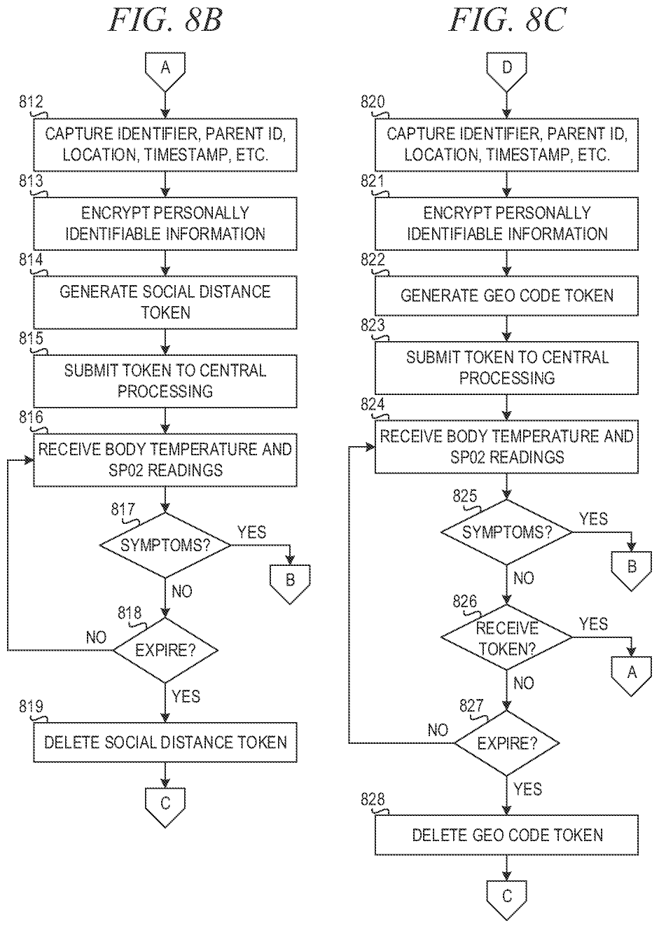

FIGS. 8A-8C are flowcharts illustrating operation of a user device for pandemic community tokenization tracing in accordance with an illustrative embodiment. With reference to FIG. 8A, operation begins (block 800), and the user device receives body temperature and SpO2 readings (block 801). Body temperature and SpO2 are examples, and there can be more sensors to measure more vitals depending on the availability and the virus might affect. The user device determines whether the user is showing symptoms of an infectious disease (block 802). The user device may determine the user is symptomatic in response to the user's body temperature being above a temperature threshold and the user's blood oxygen being below a blood oxygen threshold for a predetermined period of time or a predetermined number of consecutive readings. If the user device determines that the user is not showing symptoms in block 802, then the user device determines whether a pandemic token is received from another user device (block 803). If a pandemic token is not received, then the user device determines whether the user has visited a location that was previously visited by symptomatic or asymptomatic carriers of the infectious disease (block 804). If the user has not visited such a location, then operation returns to block 801 to monitor body temperature and SpO2 readings.

If the user device determines the user is showing symptoms in block 802, then the user device captures the identifier, geographic location, time stamp, etc. for creation of a pandemic token (block 805). The user device encrypts personally identifiable information (block 806) and generates a symptomatic pandemic token (block 807). Then, the user device submits the symptomatic pandemic token to a central processing computing device (block 808).

Next, the user device receives body temperature and SpO2 readings (block 809) and determines whether the user is showing symptoms of an infectious disease (block 810). The user device may determine whether the user has not shown symptoms for a predetermined period of time, such as two weeks. If the user device determines that the user is still showing symptoms in block 810, then operation returns to block 809 until the user is no longer symptomatic. If the user device determines that the user has not been showing symptoms for a predetermined period of time in block 810, then the user device may delete the symptomatic pandemic token and notify the central processing computing device (block 811). Thereafter, operation returns to block 801.

If the user device receives a pandemic token from another user device via short distance communication in block 803, then operation proceeds to FIG. 8B. The user device captures the identifier, geographic location, time stamp, etc. for creation of a pandemic token (block 812). The user device encrypts personally identifiable information (block 813) and generates a social distance pandemic token (block 814). Then, the user device submits the social distance pandemic token to a central processing computing device (block 815).

Next, the user device receives body temperature and SpO2 readings (block 816) and determines whether the user is showing symptoms of an infectious disease (block 817). If the user device determines that the user is showing symptoms in block 817, then operation returns to block 807 in FIG. 8A. If the user device determines that the user is not showing symptoms in block 817, then the user device determines whether a life span of the social distance pandemic token has expired (block 818). If the life span of the token has not expired, then operation returns to block 816. If user device determines that the life span of the social distance pandemic token has expired without the user showing symptoms in block 818, then the user device deletes the social distance pandemic token and notifies the central processing computing device (block 819). Thereafter, operation returns to block 801 in FIG. 8A.

Returning to FIG. 8A, if the user device determines that the user has visited a location that was previously visited by symptomatic or asymptomatic carriers of the infectious disease in block 804, then operation proceeds to FIG. 8C. The user device captures the identifier number, geographic location, time stamp, etc. for creation of a pandemic token (block 820). The user device encrypts personally identifiable information (block 821) and generates a geo code pandemic token (block 822). Then, the user device submits the geo code pandemic token to a central processing computing device (block 823).

Next, the user device receives body temperature and SpO2 readings (block 824) and determines whether the user is showing symptoms of an infectious disease (block 825). If the user device determines that the user is showing symptoms in block 825, then operation returns to block 807 in FIG. 8A. If the user device determines that the user is not showing symptoms in block 825, then the user device determines whether a pandemic token is received from another user device (block 826). If a pandemic token is received from another user device, then operation proceeds to FIG. 8B. If a pandemic token is not received in block 826, then the user device determines whether a life span of the geo code pandemic token has expired (block 827). If the life span of the token has not expired, then operation returns to block 824. If user device determines that the life span of the geo code pandemic token has expired without the user showing symptoms and without social distancing being violated in block 827, then the user device deletes the geo code pandemic token and notifies the central processing computing device (block 828). Thereafter, operation returns to block 801 in FIG. 8A.

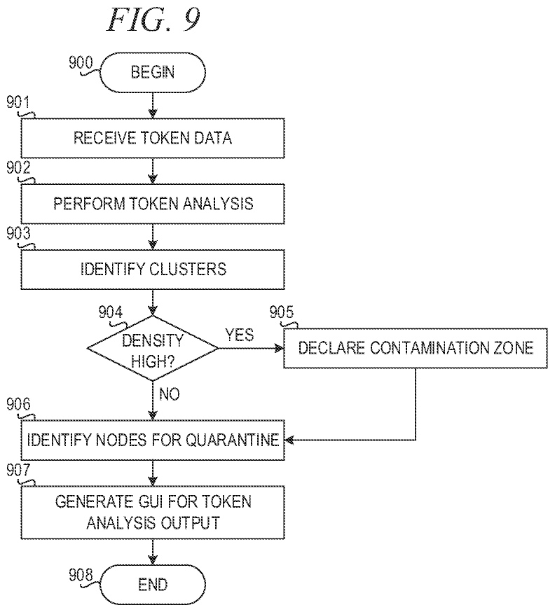

FIG. 9 is a flowchart illustrating operation of a central data processing system for pandemic community tokenization tracing in accordance with an illustrative embodiment. Operation begins (block 900), and the central data processing system receives pandemic token data from user devices (block 901). The central data processing system then performs token analysis (block 902). The central data processing system identifies clusters of related nodes (block 903). The central data processing system determines whether there is a location with a high density of symptomatic or asymptomatic carriers (block 904). If there is a high density, the central data processing system declares a contamination zone (block 905).

Thereafter, or if the central data processing system does not identify a high-density location in block 904, the central data processing system identifies nodes for quarantine (block 906). Then, the central data processing system generates a graphical user interface for token analysis output (block 907). Thereafter, operation ends (block 908).

As noted above, it should be appreciated that the illustrative embodiments may take the form of an entirely hardware embodiment, an entirely software embodiment or an embodiment containing both hardware and software elements. In one example embodiment, the mechanisms of the illustrative embodiments are implemented in software or program code, which includes but is not limited to firmware, resident software, microcode, etc.

A data processing system suitable for storing and/or executing program code will include at least one processor coupled directly or indirectly to memory elements through a communication bus, such as a system bus, for example. The memory elements can include local memory employed during actual execution of the program code, bulk storage, and cache memories which provide temporary storage of at least some program code in order to reduce the number of times code must be retrieved from bulk storage during execution. The memory may be of various types including, but not limited to, ROM, PROM, EPROM, EEPROM, DRAM, SRAM, Flash memory, solid state memory, and the like.

Input/output or I/O devices (including but not limited to keyboards, displays, pointing devices, etc.) can be coupled to the system either directly or through intervening wired or wireless I/O interfaces and/or controllers, or the like. I/O devices may take many different forms other than conventional keyboards, displays, pointing devices, and the like, such as for example communication devices coupled through wired or wireless connections including, but not limited to, smart phones, tablet computers, touch screen devices, voice recognition devices, and the like. Any known or later developed I/O device is intended to be within the scope of the illustrative embodiments.

Network adapters may also be coupled to the system to enable the data processing system to become coupled to other data processing systems or remote printers or storage devices through intervening private or public networks. Modems, cable modems and Ethernet cards are just a few of the currently available types of network adapters for wired communications. Wireless communication-based network adapters may also be utilized including, but not limited to, 802.11 a/b/g/n wireless communication adapters, Bluetooth wireless adapters, and the like. Any known or later developed network adapters are intended to be within the spirit and scope of the present invention.

The description of the present invention has been presented for purposes of illustration and description, and is not intended to be exhaustive or limited to the invention in the form disclosed. Many modifications and variations will be apparent to those of ordinary skill in the art without departing from the scope and spirit of the described embodiments. The embodiment was chosen and described in order to best explain the principles of the invention, the practical application, and to enable others of ordinary skill in the art to understand the invention for various embodiments with various modifications as are suited to the particular use contemplated. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, in a computing device, for pandemic community tokenization tracing, the method comprising:
determining whether a user of the computing device has symptoms of an infectious disease based on readings from one or more sensors;
generating a symptomatic pandemic token in response to determining the user has symptoms of the infectious disease;
transmitting the symptomatic pandemic token from the computing device to a central data processing system for analysis; and
broadcasting the symptomatic pandemic token via a short distance communication component of the computing device.

2. The method of claim 1, wherein the one or more sensors comprise a body temperature sensor and a blood oxygen sensor.

3. The method of claim 2, wherein the computing device determines the user has symptoms of the infectious disease in response to the reading from the body temperature sensor being greater than a temperature threshold and the reading from the blood oxygen sensor being less than a blood oxygen threshold for a predetermined period of time or a predetermined number of consecutive readings.

4. The method of claim 1, wherein the symptomatic pandemic token comprises a device identifier of the computing device, the readings from the one or more sensors, a location of the computing device, and a time stamp.

5. The method of claim 1, Wherein the short distance communication component uses the Industrial, Scientific, and Medical (ISM) band of frequencies.

6. The method of claim 1, wherein a transmission range of the short distance communication component is tuned based on social distancing recommendations for the infectious disease.

7. The method of claim 1, further comprising generating a social distance pandemic token in response to receiving a pandemic token from a second computing device of a second user via the short distance communication component.

8. The method of claim 7, further comprising transmitting the social distance pandemic token from the computing device to a central data processing system and broadcasting the social distance pandemic token via a short distance communication component of the computing device.

9. The method of claim 1, further comprising generating a geo code pandemic token in response to determining the user has visited a geographic location previously vised by a symptomatic or asymptomatic carrier of the infectious disease.

10. The method of claim 9, wherein the computing device determines the user has visited a geographic location previously vised by a symptomatic or asymptomatic carrier in response to receiving a pandemic token from a pandemic token communication access point.

11. The method of claim 9, further comprising transmitting the geo code pandemic token from the computing device to a central data processing system and broadcasting the geo code pandemic token via a short distance communication component of the computing device.

12. The method of claim 1, wherein the central data processing system stores the symptomatic pandemic token in a tree data structure.

13. The method of claim 12, wherein the central data processing system analyzes the tree data structure and generates a graphical user interface to output results of the analysis.

14. The method of claim 12, wherein the central data processing system identities clusters of nodes in the tree data structure.

15. The method of claim 12, wherein the central data processing system identifies declares a contamination zone responsive to identifying a high-density area.

16. The method of claim 12, wherein the central data processing system identifies nodes in the tree data structure for quarantine.

17. A computer program product comprising a computer readable storage medium having a computer readable program stored therein, wherein the computer readable program, when executed on a computing device, causes the computing device to:
determine whether a user of the computing device has symptoms of an infectious disease based on readings from one or more sensors;
generate a symptomatic pandemic token in response to determining the user has symptoms of the infectious disease;
transmit the symptomatic pandemic token from the computing device to a central data processing system for analysis; and
broadcast the symptomatic pandemic token via a short distance communication component of the computing device.

18. The computer program product of claim 17, wherein the computer readable program further causes the computing device to:
generate a social distance pandemic token in response to receiving a pandemic token from a second computing device of a second user via the short distance communication component;
transmit the social distance pandemic token from the computing device to a central data processing system; and
broadcast the social distance pandemic token via a short distance communication component of the computing device.

19. A computing device comprising:
a processor; and
a memory coupled to the processor, wherein the memory comprises instructions which, when executed by the processor, cause the processor to:
determine whether a user of the computing device has symptoms of an infectious disease based on readings from one or more sensors;
generate a symptomatic pandemic token in response to determining the user has symptoms of the infectious disease;
transmit the symptomatic pandemic token from the computing device to a central data processing system for analysis; and
broadcast the symptomatic pandemic token via a short distance communication component of the computing device.

20. The computing device of claim 19, wherein the instructions further cause the processor to:
generate a social distance pandemic token in response to receiving a pandemic token from a second computing device of a second user via the short distance communication component;
transmit the social distance pandemic token from the computing device to a central data processing system; and
broadcast the social distance pandemic token via a short distance communication component of the computing device.
